(19) United States
(12) Patent Application Publication
(10) Pub. No.: US 2025/0064549 A1
(43) Pub. Date: Feb. 27, 2025

(54) INSTRUMENT HOLDER

(71) Applicant: Hamilton Surgical Instruments LLC, Louisville, KY (US)

(72) Inventor: Jeremy Alan Hamilton, Louisville, KY (US)

(21) Appl. No.: 18/815,314

(22) Filed: Aug. 26, 2024

Related U.S. Application Data
(60) Provisional application No. 63/578,803, filed on Aug. 25, 2023.

(51) Int. Cl. A61B 90/50; A61B 17/02

(52) U.S. Cl. CPC A61B 90/50; A61B 17/02

(57) ABSTRACT

The specification discloses a novel instrument holder including a vertical support for engaging an operating table mount. The vertical support is includes splines or a polygonal shaft that is be engaged by a tension bar having complementary apertures and slots for securing and engaging an instrument for use in a surgical procedure.

### BACKGROUND OF THE INVENTION

The system described herein relates generally to an apparatus for securing an instrument to an operating platform, and more particularly to a system and apparatus for adjustably securing a retractor instrument to an operating table. The embodiments described herein feature a novel platform to mount, secure and adjust the positioning of a retractor for a plurality of surgical uses.

In surgical applications a variety of tools and instruments are employed by surgeons to perform the requisite tasks required to complete a successful surgical procedure. Many of these tools are highly specialized and have a single function or purpose to assist in the surgery. For example, when orthopedic surgeons perform anterior hip arthroplasty a femoral hook is provided to assist in exposure of the femur, which is necessary to provide access to the surgical space throughout the procedure. In conjunction with the femoral hook a retractor, often a retractor called a “narrow Hohmann” is typically employed to hold back soft tissue around the surgical site, thereby providing access to the site during the procedure.

In exemplary anterior hip arthroplasty procedures a medical professional must carefully position at least these two instruments, the femoral hook and the retractor, such that the surgical site remains open for access. The femoral hook is often attached to a surgical table via a horizontal support so that it remains in place. However, the retractor usually must be held by a medical professional, since pressure must be maintained on the retractor in order to keep the tissue pulled out of the way. Furthermore, this retractor typically must be repositioned throughout the procedure, thus requiring the use of a hand, or a pair of hands. This naturally requires additional staffing and reduces the efficiency of the surgical staff.

Accordingly, there is a need in the art for a retractor holder that is simple, easy to operate, efficient to assembly, and interoperable with existing surgical tables to aid in the efficiency of certain surgeries requiring tissue retraction.

### SUMMARY OF THE INVENTION

In various aspects and embodiments the invention described herein provides a system for holding a retractor during a surgical procedure, including a vertical support and a concomitant horizontal tension bar that engages the vertical support. The embodiments described herein are capable of being used in conjunction with a commonly employed surgical table, particularly a surgical table adapted for use in anterior hip arthroplasty that includes a horizontal support securely mounted to the table for mounting instruments. A wide variety of uses of the described embodiments may be implemented utilizing a variety of surgical tools without departing from the teachings of this specification.

In some aspects and embodiments a vertical support for engaging a retractor or similar surgical instrument is provided having a shaped cross-section for engaging a complimentary aperture in a tension bar. In some embodiments the vertical support is capable of permitting the tension bar to be adjusted vertically to provide varying heights for engagement of the surgical instrument.

In various embodiments and aspects the tension bar includes a shaped aperture, or a plurality thereof disposed at various angles and spacings along the tension bar, for engaging the vertical support and positioning the tension bar proximate the surgical site.

In some embodiments the tension bar includes a plurality of retractor positioning slots, bounded by a plurality of slot walls that extend both above and below the tension bar, for engaging and holding an end of a retractor for use in the surgical procedure.

As used herein for purposes of the present disclosure, the term “retractor” can be used to refer to any of a broad variety of surgical retractors or other tools adapted for use in surgical procedures. Any of a wide variety of retractors may be employed with the concepts and embodiments described without limiting the scope of the invention.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other advantages and features, which characterize the embodiments, are set forth in the claims appended hereto and form a further part thereof. However, for a better understanding of the embodiments and of the advantages and objectives attained through their use, reference should be made to the Drawing Figures and to the accompanying specification, in which there are described exemplary embodiments. This summary is merely provided to introduce a selection of concepts that are further described below in the detailed description, and is not intended to identify key or essential features of the claimed subject matter, nor is it intended to be used in limiting the scope of the claimed subject matter.

### BRIEF DESCRIPTION OF THE DRAWING FIGURES

In the drawings, like reference characters generally refer to the same parts throughout the different views. Also, the drawings are not necessarily to scale, emphasis instead generally being placed upon illustrating the principles of the invention.

FIG. 1 depicts various views and features of a surgical instrument holder in an operating environment in accordance with various embodiments;

FIG. 2 is a perspective view of an instrument holder assembly in accordance with various embodiments;

FIG. 3 is a perspective view of an instrument holder assembly in accordance with various embodiments;

FIG. 4 depicts several views of a tension bar in accordance with various embodiments;

FIG. 5 is a perspective view of an instrument holder in accordance with various embodiments;

FIG. 6 is a perspective view of an instrument holder in accordance with various embodiments;

FIG. 7 depicts several views of an instrument holder in accordance with various embodiments;

FIG. 8A depicts several views of an instrument holder in accordance with various embodiments;

FIG. 8B depicts several views of an instrument holder in accordance with various embodiments;

FIG. 9A depicts several views of an instrument holder in accordance with various embodiments;

FIG. 9B depicts several views of an instrument holder in accordance with various embodiments;

FIG. 9C depicts several views of an instrument holder in accordance with various embodiments;

FIG. 10A depicts several views of an instrument holder in accordance with various embodiments;

FIG. 10B depicts several views of an instrument holder in accordance with various embodiments;

FIG. 11A depicts several views of an instrument holder in accordance with various embodiments;

FIG. 11B depicts several views of an instrument holder in accordance with various embodiments;

FIG. 11C depicts several views of an instrument holder in accordance with various embodiments;

FIG. 12A depicts several views of an instrument holder in accordance with various embodiments;

FIG. 12B depicts several views of an instrument holder in accordance with various embodiments; and

FIG. 12C depicts several views of an instrument holder in accordance with various embodiments.

### DETAILED DESCRIPTION OF THE PREFERRED EMBODIMENT(S)

Numerous variations and modifications of the apparatuses and embodiments of the disclosed invention will be apparent to one of ordinary skill in the art, as will be readily seen from the detailed description of the system set forth below. Therefore, the apparatus is not limited to the specific implementations discussed herein.

Furthermore, the embodiments and aspects detailed in this specification will focus on the implementation of an apparatus, system and operational techniques for a surgical instrument holder. While the embodiments described herein will refer generally to implementation of a holder for a surgical instrument, specifically a retractor, one of ordinary skill will recognize that the invention may be practiced with a broad array of equivalent assemblies and constructions without departing from the scope thereof.

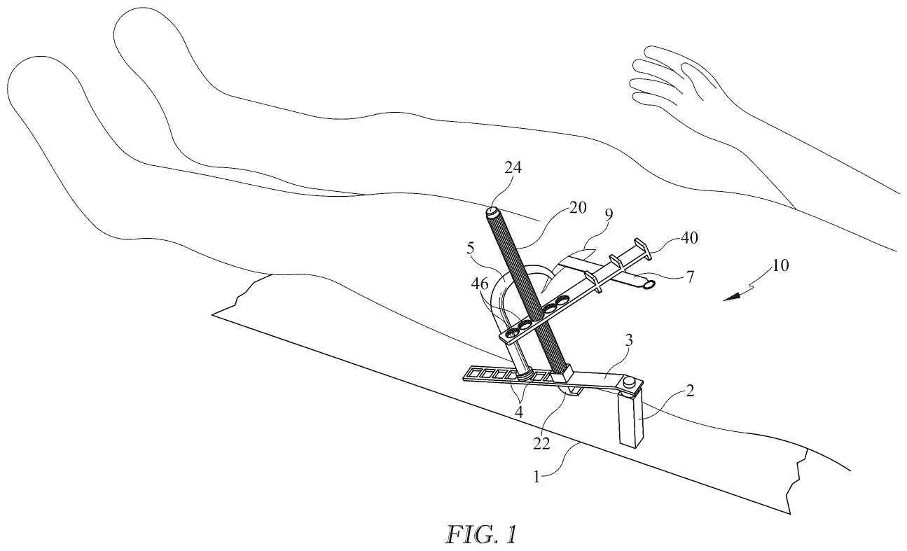

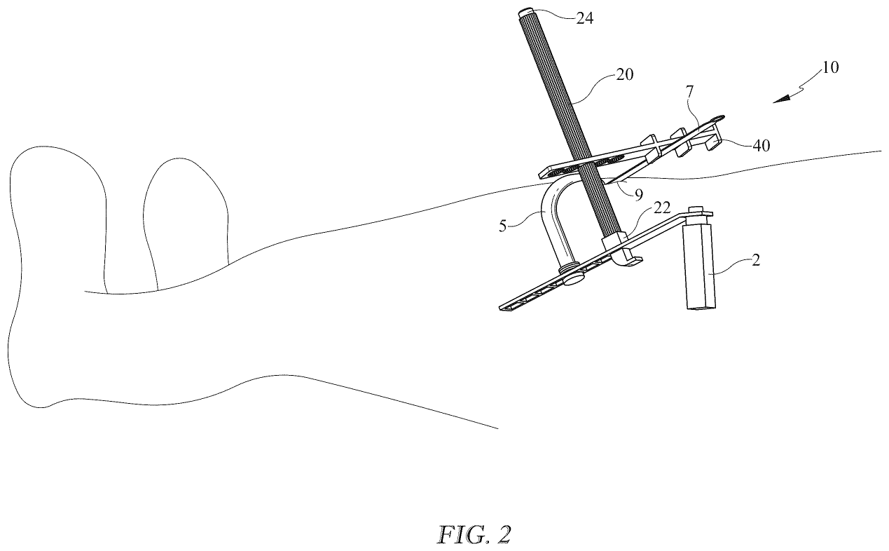

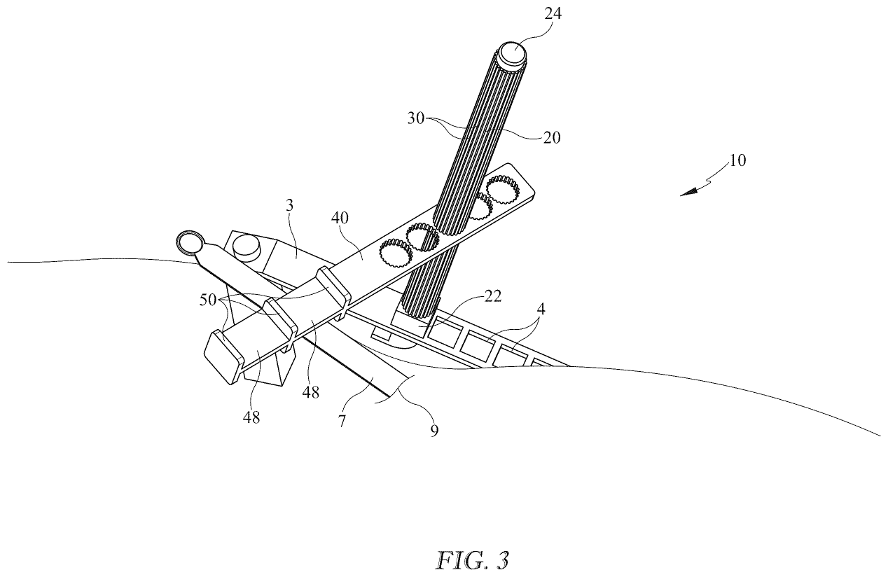

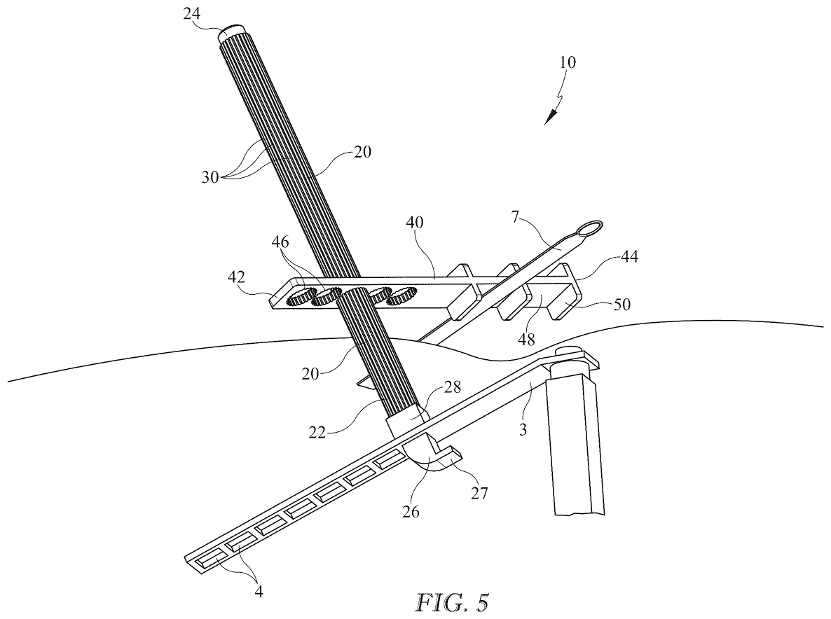

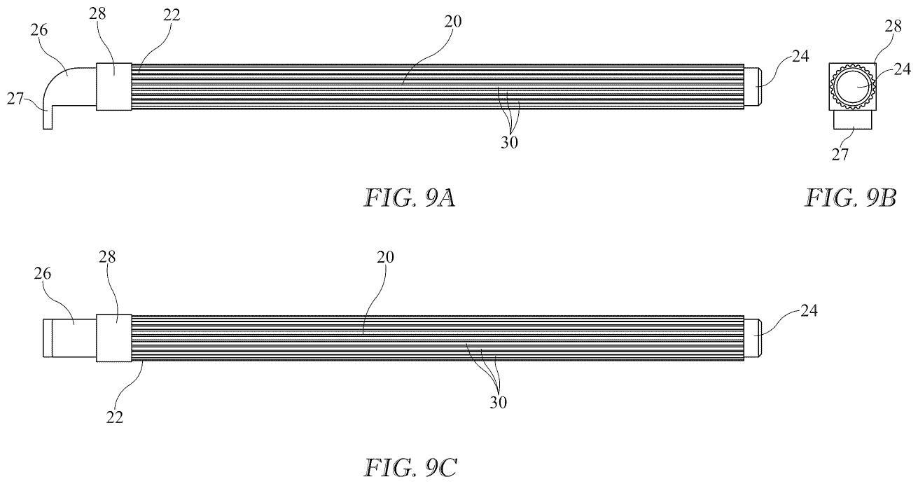

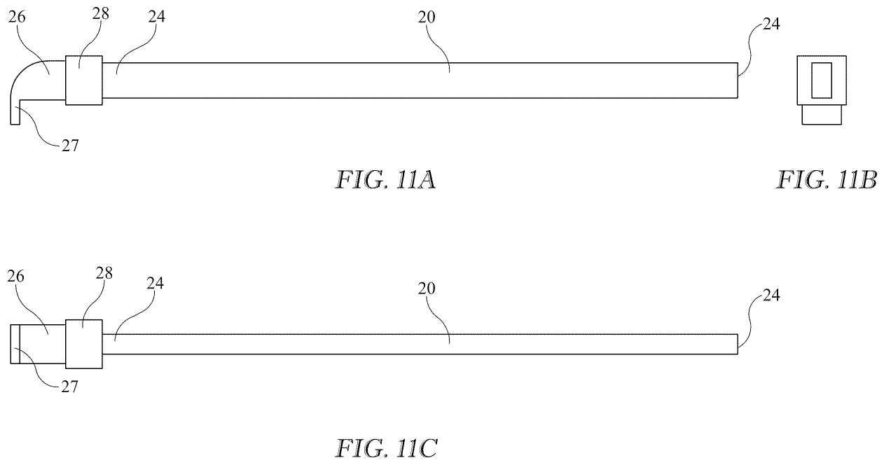

Referring now to FIGS. 1-12C and in accordance with various aspects and embodiments, herein is described a novel instrument holder system 10. For example, the embodiments described herein are capable of being employed and/or adapted for use in a surgical system utilizing a retractor, and more specifically a “narrow Hohmann” retractor as typically used in anterior hip arthroplasty procedures. However, many other embodiments and uses may be employed implementing the disclosed system and apparatus utilizing a variety of surgical systems and retractor types in many different surgeries without departing from the teachings of this specification. Throughout this specification the system described will be utilized in the environment of an anterior hip arthroplasty surgery, and the generic term “retractor” will be used for ease of explication. However, the disclosed embodiments may be used in many other surgical applications, in fact nearly any other surgical procedure that requires the use of a retractor that is held in place.

In some aspects and embodiments as best depicted in FIGS. 1-5 an instrument holder apparatus 10 is designed to be employed within a surgical environment that includes a surgical table 1 having a vertical mount 2, and a horizontal support 3 for engaging, securing and supporting a variety of instruments commonly used in surgical procedures. In exemplary anterior hip arthroplasty surgeries, often a “Hana®” surgical table is employed, that includes a horizontal support 3 that is typically called a “femoral hook support” 3. The femoral hook support 3 includes a plurality of apertures 4 spaced along its length through which an end of a femoral hook 5 may be positioned. The other end of femoral hook 5 is positioned in the surgical site by the surgeon to assist with femoral exposure during the surgical procedure.

The instrument holder 10 includes a vertical support bar 20 that is secured to horizontal support 3 that extends generally upwardly to support and engage a tension bar 40, as seen in FIGS. 1-6 for example. It should be noted that in some exemplary embodiments all components of system 10 described herein are manufactured of 316 stainless steel or the equivalent, suitable for use in surgical applications and capable of being autoclaved and reused. However, a wide variety of stainless alloys, and other metallic and non-metallic materials may be used in constructing the components of the various embodiments. In some embodiments carbon steel, aluminum, or event high-strength plastics can be used to construct the disclosed components of system 10.

Referring again to FIGS. 1-6 vertical support bar 20 has top 24 and bottom 22 ends and in some embodiments is generally straight and includes a shaped portion 26 to engage one of the plurality of apertures 4 of femoral hook support 3 so that the vertical support bar 20 bottom 22 end fits snugly into one of the apertures 4. In some embodiments bottom end 22 of support bar 20 includes a hook or j-hook 27 enabling quick insertion of bottom end 22 into aperture 4. Furthermore, bottom end 22 of support bar 20 may include a flange or stop 28 above hook 27 to prevent vertical support bar 20 from slipping through apertures 4 of horizontal support 3. While bottom end 22 of vertical support 20 is depicted as having a j-hook that engages apertures 4 of horizontal support 3 for positive placement as instrument holder 10 is assembled and positioned, it will be recognized that a variety of equivalent positioning aids may be incorporated on bottom end 22 of vertical support 20.

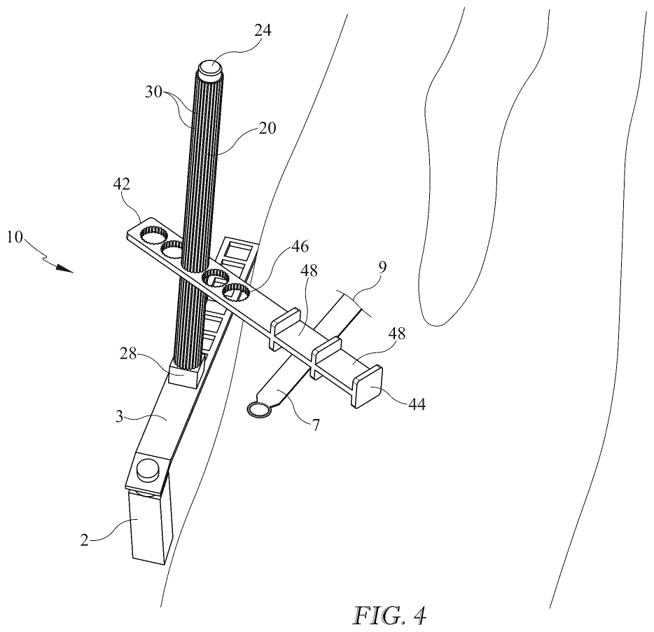

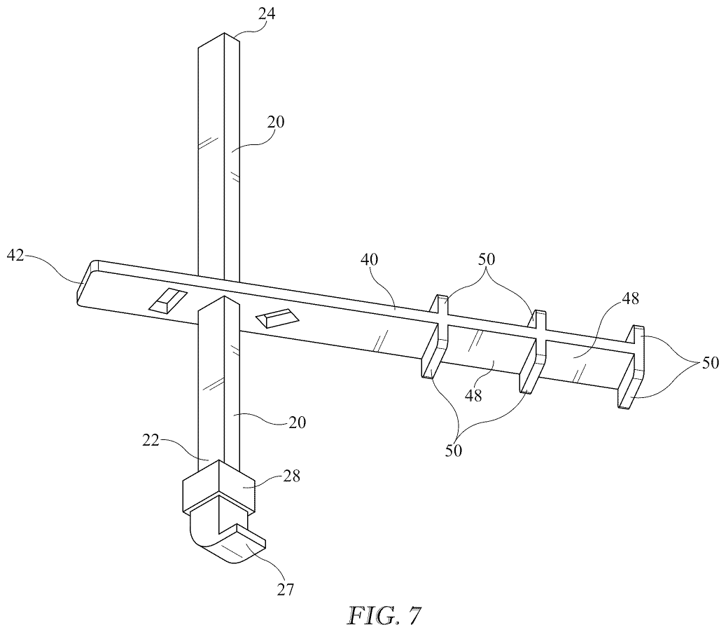

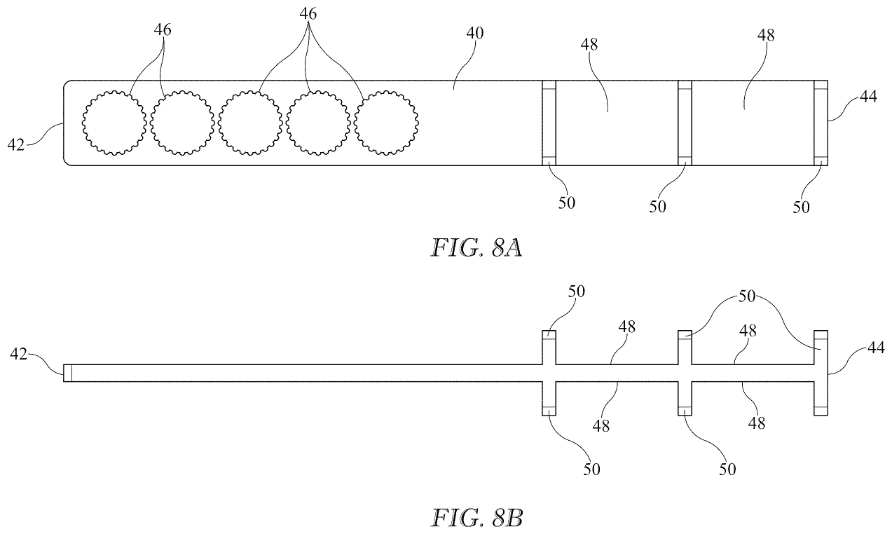

Referring to exemplary embodiments in FIGS. 4, 5 and 6 for example, vertical support 20 may be constructed in the shape of a generally cylindrical shaft and include a plurality of splines 30 spaced circumferentially along the length thereof for engaging a tension bar 40, as discussed below. Furthermore, splines 30 may be equally spaced around vertical support 20, as discussed further herein below. In other embodiments as best depicted in FIGS. 7 and 8, vertical support 20 may have a square or rectangular cross-section, as necessary to engage a complementary aperture as described below.

Referring now to FIGS. 1-7 and in various aspects the instrument holder system includes a tension bar 40 that is designed to engage and be secured to vertical support 20, and further to engage with and position a retractor 7. The tension bar 40 has first 42 and second 44 ends, and includes at least one shaped aperture 46 there through, positioned proximate first end 42 thereof. Aperture or apertures 46 are shaped to be complementary to the shape of vertical support 20 so tension bar 40 aperture 46 can engage and be moved upwardly or downwardly along vertical support 20 for an optimal tension bar 40 vertical position. While a wide variety of shapes and sizes may be used for tension bar apertures 46 and vertical support 20, in some exemplary embodiments as depicted in FIGS. 7, 8 and 11A-11C vertical support 20 may have a square cross-section, or a generally rectangular cross-section, each with our without chamfered edges. Furthermore, in certain embodiments as depicted in FIGS. 1-6 and 9A-9C vertical support 20 may be generally cylindrical and include a plurality of splines 30 around the circumference thereof and along at least a portion of the length of vertical support 20. Accordingly, tension bar 40 apertures 46 may be shaped to engage a plurality of vertical support 20 cross-sectional shapes. In these embodiments tension bar 40 can be positioned in a plurality of positions with respect to surgical site 9, since splines 30 act as an indexing apparatus, permitting a surgeon to move tension bar 40 around vertical support 20 by one on more spline widths for precise positioning thereof.

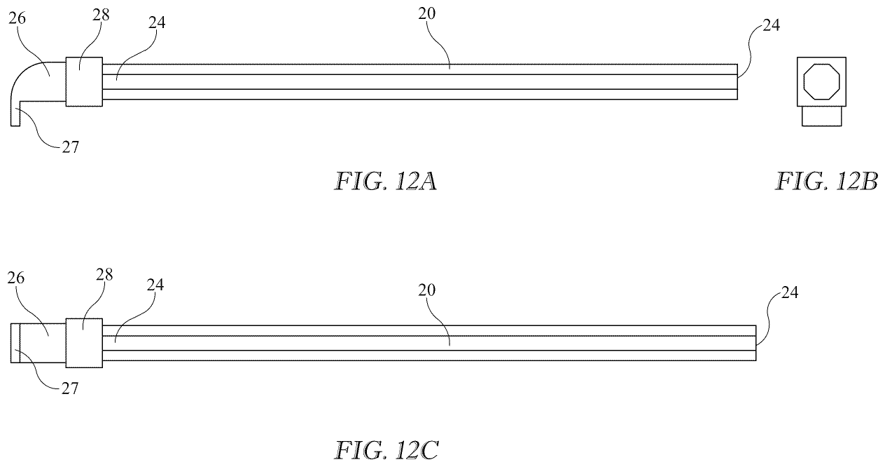

For example, and as best depicted in FIGS. 1-6, 8A and 8B, tension bar 40 apertures 46 may be generally circular and have a plurality of grooves 48 disposed around the circumference of aperture 46. For apertures 46 having this shape, vertical support 20 can include a plurality of splines 30, or even have a polygonal cross-section wherein the vertices of the polygon are sized to engage grooves 48 spaced around apertures 46. In some exemplary embodiments, apertures 46 may include 24 evenly spaced grooves around the circumference thereof, such that vertical support 20 can have a cross-section that is square, hexagonal, octagonal, or even 12 or 16 sided and still engage apertures 46 as long as the sides 32 have the correct length to engage grooves 48. FIGS. 12A-12C depict vertical support 20 having an exemplary octagonal cross-section for use with a complementary aperture 46 of tension bar 40.

Accordingly, horizontal tension bar 40 apertures 46 can engage vertical support 20 and tension bar 40 can be positioned at any point along vertical support 20 for optimal positioning. Since tension bar 40 apertures 46 are disposed proximate first end 42 of tension bar 40, the weight of tension bar 40 will hold it in place once it is vertically positioned along vertical support 20, in a cantilevered fashion.

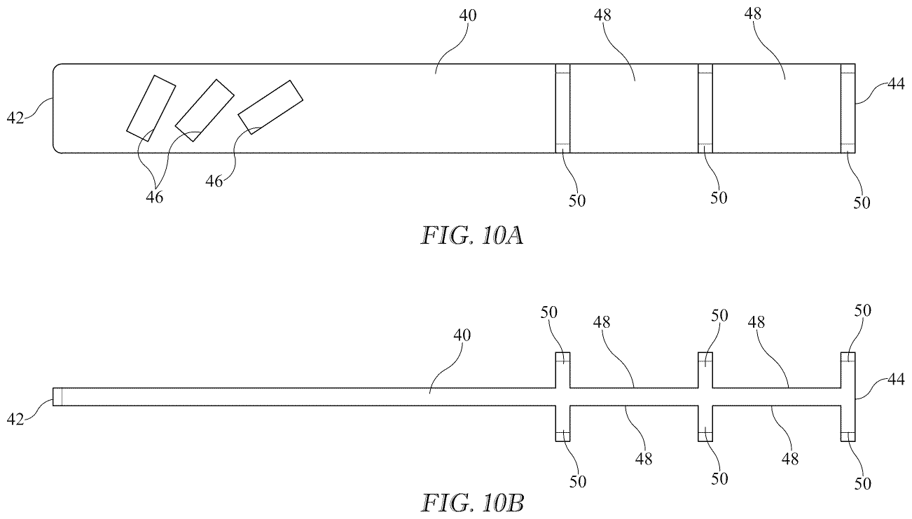

In accordance with further aspects and embodiments and as depicted in FIGS. 7, 10A and 10B, for example, a plurality of elongated or slot type apertures 46 may be provided through and proximate first end 42 of tension bar 40. Each aperture 46 may have a different orientation or angle with respect to the length of tension bar 46 so that tension bar 40 may be positioned at various angles with respect to vertical support 20. In these embodiments, vertical support 20 has a cross-section that is complementary to apertures 46, for example rectangular with chamfered edges. In some exemplary embodiments apertures 46 may be provided at 30, 45 and 60 degrees relative to a longitudinal axis of tension bar 40, although a wide variety of aperture 46 orientations may be used without departing from the scope of the disclosed embodiments.

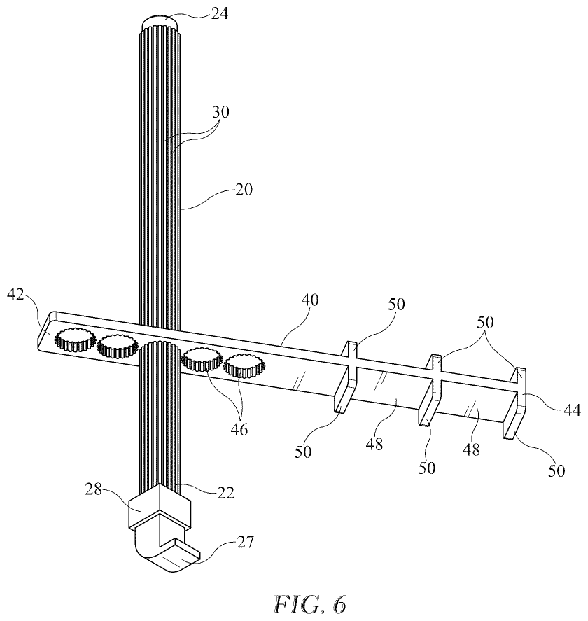

In further aspects and embodiments as best seen in FIGS. 6, 7, 8A, 8B, 10A and 10B tension bar 40 includes a plurality of retractor positioning slots 48 along its length proximate second end 44 that engage an end or portion of retractor 7, as best depicted in FIG. 1. The retractor slots 48 may be bounded by a plurality of positioning slot walls 50 that depend vertically both above and below tension bar 40 such that the orientation of tension bar 40 with respect to vertical support 20 is irrelevant when assembling system 10. This feature of system 10 permits an end of retractor 7 to be positioned in a one of said plurality of slots 48 and be slightly adjustable side to side while slot walls 50 provide a hard stop in one direction or the other as retractor 7 is subjected to tension during the procedure. While the embodiments depicted show two positioning slots 48, one of ordinary skill will understand that a plurality of tensioning slots 48 and slot walls 50 may be provided along the length of tension bar 40 without departing from the scope of the disclosed embodiments.

In the context of utilizing system 10, operation a medical professional inserts retractor 7 end into a surgical site 9 to retract tissue as required by the procedure being performed, whereupon, the other end of retractor 7 is advantageously positioned in a tension bar 40 slot 48, and thus held in place by the spring tension of retractor 7 pushing upwardly on tension bar 40. The vertical positioning of tension bar 40 and thus retractor 7 and the force supplied to retractor 7 can then be adjusted by changing the vertical position of tension bar 40 along vertical support 20. Furthermore, the angle of tension bar 40, and thus tension bar 40 slot 48 angles or orientations along with retractor 7 position, can be changed by selecting a different tension bar 40 aperture 46 to engage vertical support 20.

While several embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It is to be understood that the embodiments are not limited in its application to the details of construction and the arrangement of components set forth in the description or illustrated in the drawings. The invention is capable of other embodiments and of being practiced or of being carried out in various ways. Unless limited otherwise, the terms “connected,” “coupled,” “in communication with,” and “mounted,” and variations thereof herein are used broadly and encompass direct and indirect connections, couplings, and mountings. In addition, the terms “connected” and “coupled” and variations thereof are not restricted to physical or mechanical connections or couplings.

While the present embodiments have been shown and described herein in what are considered to be exemplary aspects of the system, illustrating the results and advantages over the prior art obtained, the system is not limited to those specific embodiments. Thus, the forms of the system shown and described herein are to be taken as illustrative only and other embodiments may be selected without departing from the scope thereof, as set forth in the claims appended hereto.

I claim:

1. A system for securing and positioning a retractor for use with a surgical table having a vertical mount and a horizontal support having a plurality of instrument apertures therein comprising:
a vertical support bar for engaging a one of said instrument apertures; and
a tension bar adjustably secured to said vertical support bar, said tension bar positioned in a generally horizontal orientation and having a plurality of retractor positioning slots arranged thereon for engaging said retractor.

2. The system of claim 1 wherein said tension bar comprises:
a plurality of apertures therein for engaging said vertical support bar.

3. The system of claim 2 wherein said plurality of apertures are generally cylindrical and include a plurality of grooves around the aperture for engaging said vertical support bar at a plurality of different angles.

4. The system of claim 1 wherein said tension bar comprises:
a plurality of positioning slot walls extending above and below said tension bar for retaining and positioning said retractor along said tension bar.

5. The system of claim 1 where said vertical support comprises:
a bottom end shaped to engage said horizontal support instrument apertures, said bottom end having a stop that prohibits said vertical support from passing through said instrument apertures.

6. The system of claim 1 where said vertical support comprises:
a bottom end having a j-hook thereon to engage said horizontal support instrument apertures.

7. The system of claim 1 where said vertical support comprises:
a generally cylindrical shaft having a plurality of splines along a length thereof for engaging the apertures of said tension bar.

8. The system of claim 1 wherein said vertical support comprises:
a shaft having a generally polygonal cross section sized to engage the apertures of said tension bar.

9. The system of claim 8 wherein said vertical support has an octagonal cross-section for engaging complementary apertures of said tension bar.

10. The system of claim 1 wherein said vertical support and said tension bar are comprised of 316 stainless steel.

11. A system for securing and positioning a retractor for use with a surgical table having a vertical mount and a horizontal support having a plurality of instrument apertures therein comprising:
a vertical support bar for engaging a one of said instrument apertures; and
a tension bar adjustably secured to said vertical support bar, said tension bar having a plurality of angled apertures therein for engaging said vertical support bar and having a plurality of retractor positioning slots arranged thereon for engaging said retractor.

12. The system of claim 11 wherein said plurality of angled apertures include at least three apertures arranged at 30, 45 and 60 degrees respectively for engaging said vertical support bar at a plurality of different angles.

13. The system of claim 11 wherein said tension bar comprises:
a plurality of positioning slot walls extending above and below said tension bar for retaining and positioning said retractor along said tension bar.

14. The system of claim 11 where said vertical support comprises:
a bottom end shaped to engage said horizontal support instrument apertures, said bottom end having a stop that prohibits said vertical support from passing through said instrument apertures.

15. The system of claim 10 wherein said vertical support and said tension bar are comprised of 316 stainless steel.
